(19) United States
(12) Patent Application Publication
(10) Pub. No.: US 2015/0342497 A1
(43) Pub. Date: Dec. 3, 2015

(54) METHOD AND APPARATUS FOR ACQUIRING OF SIGNALS FOR ELECTRICAL IMPEDANCE

(71) Applicant: TIMPEL S.A., São Paulo -SP (BR)

(72) Inventors: Edward Lazo Maktura, São Paulo (BR); Celso Gonzalez Lima, Rio de Janeiro (BR)

(21) Appl. No.: 14/760,192

(22) PCT Filed: Jan. 9, 2013
(86) PCT No.: PCT/BR13/00008

§ 371 Date: Jul. 9, 2015

(51) Int. Cl. A61B 5/053; A61B 5/00

(52) U.S. Cl. CPC A61B 5/0536; A61B 5/7275; A61B 5/7228

(57) ABSTRACT

The method of the disclosure comprises the stages of injecting an alternating AC signal into at least one electrode positioned on the skin of the patient in a region of interest, measuring the signal induced into at least one electrode, extracting at least an envelope of the signal measured and determining the difference between the envelope and a reference signal. The envelope may be extracted by demodulating the signal measured, and this demodulation may be carried out by way of an associated rectifier diode, in a preferred embodiment, and an RC filter. The reference signal may be an envelope corresponding to the demodulated signal of another electrode, the injected signal, ground or a fixed voltage value.

### CROSS-REFERENCE TO RELATED APPLICATIONS

This application is a national phase entry under 35 U.S.C. §371 of International Patent Application PCT/BR2013/000008, filed Jan. 9, 2013, designating the United States of America and published in Portuguese as International Patent Publication WO 2014/107772 A1 on Jul. 17, 2014, the disclosure of which is hereby incorporated herein in its entirety by this reference.

### TECHNICAL FIELD

This disclosure pertains to the field of electrical impedance tomography and, more particularly, to the treatment of signals measured through the electrodes positioned in contact with the patient's skin.

### BACKGROUND

Electrical impedance tomography is a known and widely used technique consisting of positioning a plurality of electrodes around a region of the patient's body, such as, for example, his chest, and injecting electrical excitation signals accompanied by measuring the other electrodes of the induced signals so as to generate a map indicative of impedance, whereby it is possible to determine respiratory and hemodynamic parameters of the patient, and to generate images that represent these parameters.

In each measurement cycle, the excitation electrodes are sequenced so as to include the majority or all the electrodes installed around the region of interest, systems with 16 or 32 electrodes being typically used. Generally, 20 to 50 measurement cycles per second (images per second) are generated, and alternating excitation signals with a frequency typically between 10 kHz and 2.5 MHz are used. These characteristics enable Electrical Impedance Tomography apparatuses to capture well the main characteristics of hemodynamics and breathing of human patients, which are phenomena with a frequency typically of the order of 1 to 4 Hz in the case of heartbeats and lower than 1 Hz in breathing.

According to known techniques, an alternating electrical signal is injected between at least two electrodes, also inducing alternating signals, which are measured in the other electrodes. Through the differences between the injected signal and the signals measured, and between the very signals measured, followed by digital signal processing techniques, a map representative of the impedances of the region of interest is constructed. It is fundamental to note that this technique presupposes that the induced alternating signals that were measured in the electrodes are synchronized with each other and also with the injected signal, which rarely occurs since the materials traversed by each of these signals do not have a uniform composition, and may present impedance variations both in the resistive component and in the reactive component. Accordingly, the phases of the signals measured should be offset by the equipment for the construction of impedance maps.

Given the fact that each measurement cycle comprises a plurality of stages, these differing in that the injection is made in different electrodes at each stage, the equipment must continuously adjust the phases of the signals measured during the course of the cycle, meaning Electrical Impedance Tomography apparatuses are required to be significantly complex, both from the perspective of circuit tuning and in harmony with processing capacitance.

Moreover, the adjustment values of the phases depend on the frequency of the injected signal, making this adjustment complex when using more than one frequency to generate the impedance maps.

### BRIEF SUMMARY

In view of the above, the objective of this disclosure is to provide a method and an apparatus for carrying out electrical impedance tomographies, by injecting a high-frequency alternating current into successive pairs of electrodes, measuring the induced voltages in the other electrodes and extracting low-frequency signals indicative of hemodynamic and breathing activities.

Another objective consists of providing a method of extracting low-frequency signals that dispenses with the phase adjustment inherent in traditional methods of Electrical Impedance Tomography, which currently presupposes synchronization between the induced and injected signals.

Another objective consists of providing a method and apparatus in which the change of frequency of the injected signal does not imply adjustments in the extraction device of low-frequency signals.

The above objectives, as well as others, are achieved by the disclosure through a method that comprises the following stages:
Injecting an alternating AC signal (carrier), through electrodes positioned around the region of interest of a patient's body;
Measuring the induced AC signal in at least one electrode;
Demodulation of the AC signal measured for extracting at least an envelope, by means of rectification and optional filtering;
Identifying the difference between the demodulated signal and a reference signal, so as to provide data for determining the impedance map corresponding to the region of interest.

According to another characteristic of the disclosure, after rectification, at least an envelope of the AC signal measured is extracted.

According to another characteristic of the disclosure, the extraction of the envelope is provided by filtering the demodulated signal.

According to another characteristic of the disclosure, the method additionally comprises identifying the difference between the envelope and a reference signal.

According to another characteristic of the disclosure, the reference signal is the signal measured in another electrode.

According to another characteristic of the disclosure, the reference signal is the injected signal.

According to another characteristic of the disclosure, the reference signal is earth or a fixed voltage value.

According to another characteristic of the disclosure, the frequency of the carrier may be varied depending on the anatomical characteristics of the patient.

According to another characteristic of the disclosure, the signal is injected into one electrode or simultaneously into more than one electrode.

According to another characteristic of the disclosure, the signal is injected successively into electrodes positioned on the body so as to complete a measurement cycle around the region of interest.

According to another characteristic of the disclosure, at least one electrode is not used for injecting the signal so as to complete the measurement cycle around the region of interest.

According to another characteristic of the disclosure, the difference between the envelope and the reference signal is provided by subtracting the signals through an analogical electronic circuit.

According to another characteristic of the disclosure, the difference between the envelope and the reference signal is provided by subtracting the signals through digital signal processing.

According to another characteristic of the disclosure, the measurements of the induced signals in the electrodes occur simultaneously.

According to another characteristic of the disclosure, the apparatus for acquiring electrical impedance tomography signals comprises at least a current source, at least a demodulation circuit per channel, at least a circuit for analogical subtraction between the demodulated signal and a reference signal and at least a circuit for conversion from analogical to digital (A/D) from that resulting from the subtraction between the demodulated signal and a reference signal.

According to another characteristic of the disclosure, the current source generating the injection signal is of the single-ended type.

According to another characteristic of the disclosure, the current source generating the injection signal is of the bipolar type.

According to another characteristic of the disclosure, each electrode has a corresponding current source.

According to another characteristic of the disclosure, the circuit's fixed capacitances are offset by at least a Negative Impedance Converter (NIC) circuit.

According to another characteristic of the disclosure, the fixed capacitances are the input capacitances of the operational amplifier.

According to another characteristic of the disclosure, the fixed capacitances are analog switch capacitances.

According to another characteristic of the disclosure, the demodulation circuit comprises a rectifier diode.

According to another characteristic of the disclosure, the demodulation circuit comprises a rectifier diode and an RC filter.

According to another characteristic of the disclosure, the demodulation circuit provides an offset for the rectifier diode, be it a positive or a negative offset.

According to another characteristic of the disclosure, each channel receives signals corresponding to more than one electrode.

### DESCRIPTION OF THE DRAWINGS

Other characteristics and advantages of this disclosure will become more obvious in the description of a preferred embodiment, given as an example as opposed to imposing limits, and of the drawings referring thereto, wherein:

FIG. 1 schematically illustrates the positioning of the electrodes on the surface of a region of interest of a patient.

FIG. 2 illustrates the circuit that treats the signals captured by two electrodes.

FIGS. 3 to 8 illustrate the signal wave forms, corresponding to various stages of the treatment of the signal captured by two electrodes.

FIG. 9 exemplifies a circuit endowed with additional characteristics, to carry out the processing of the signal captured by one of the electrodes.

### DETAILED DESCRIPTION

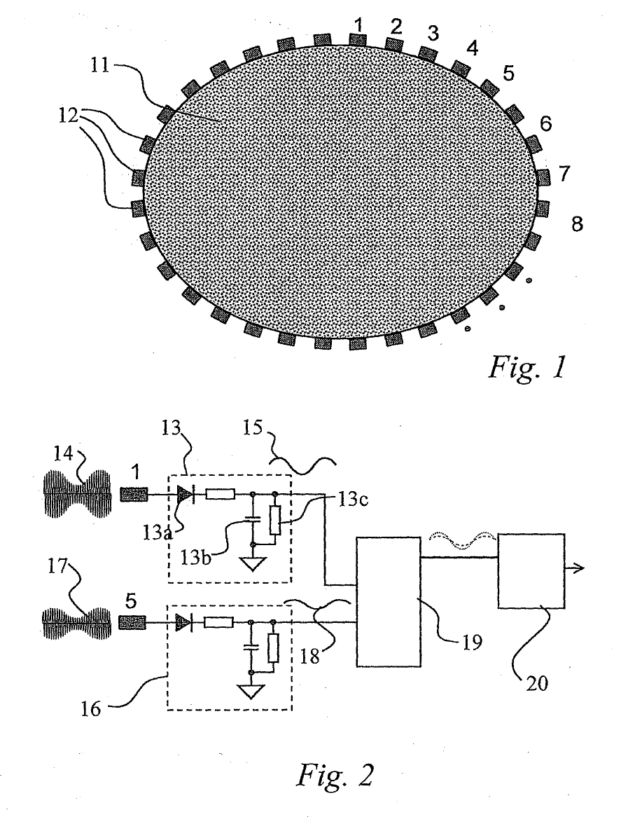

FIG. 1 schematically illustrates the application of a plurality of electrodes 12 around a region of interest of a patient II, according to known techniques. The present example uses 32 electrodes, referenced in sequence with the numbers 1, 2, 3, etc., it being understood that the disclosure is applicable to a different number of electrodes, which may be distributed uniformly or otherwise.

According to known principles expounded in prior documents, such as, for example, U.S. Pat. No. 4,617,939, U.S. Pat. No. 5,311,878, U.S. Pat. No. 5,626,146, U.S. Pat. No. 5,807,251 and U.S. Pat. No. 5,919,142, an excitation current is injected into at least one of the electrodes and then the voltages induced into the other electrodes are measured. In the most commonly used prior embodiments, a current is applied between a pair of adjacent electrodes and the voltage between the other pairs of adjacent electrodes is measured.

Even though acceptable results have been obtained with the technique referred to above, arrangements can be used in which the excitation current is applied to non-adjacent electrodes, one, two or more electrodes being spaced between the excitation electrodes, and the same may occur with the measurement electrodes. The arrangements are designated “interleave.”

For interleave three, for example, the apparatus injects an alternating AC current into electrodes 1 and 5 and takes the simultaneous measurement of the induced signals in the other pairs of electrodes (2-6; 3-7; 4-8; 6-9; 7-10, etc.).

Next, it injects an AC current into electrodes 2 and 6 and again takes the simultaneous measurement of the induced signals in the other pairs of electrodes (1-5; 3-7; 4-8; 6-9; 7-10, etc.), and so on and so forth, until completing a measurement cycle around the region of interest.

FIG. 2 schematically shows the elements of the circuit related to the treatment of the signals measured by electrodes 1 and 5. As illustrated, the alternating signal 14 measured by the electrode 1 is introduced into the set of components 13, which comprises a rectifier diode 13a and the RC filter comprising the capacitor 13b and the resistor 13c. The output of the set 13 is an envelope 15 of the original signal, which, in the present example, is the upper envelope. The signal 17 captured by the electrode 5 is subjected to identical treatment by the set 16, resulting in the signal 18, which is the envelope superior of the originally measured signal 17. This signal is used as reference in the following signal treatment stage, performed by the instrumentation amplifier (with or without programmable gain) 19, this stage consisting of identifying the difference between signals 15 and 18, obtained, for example, by subtracting the reference signal 18 from the signal 15. The result of this subtraction is then sent to the converter A-D 20, the digitalized output being sent for processing by a controller (not illustrated).

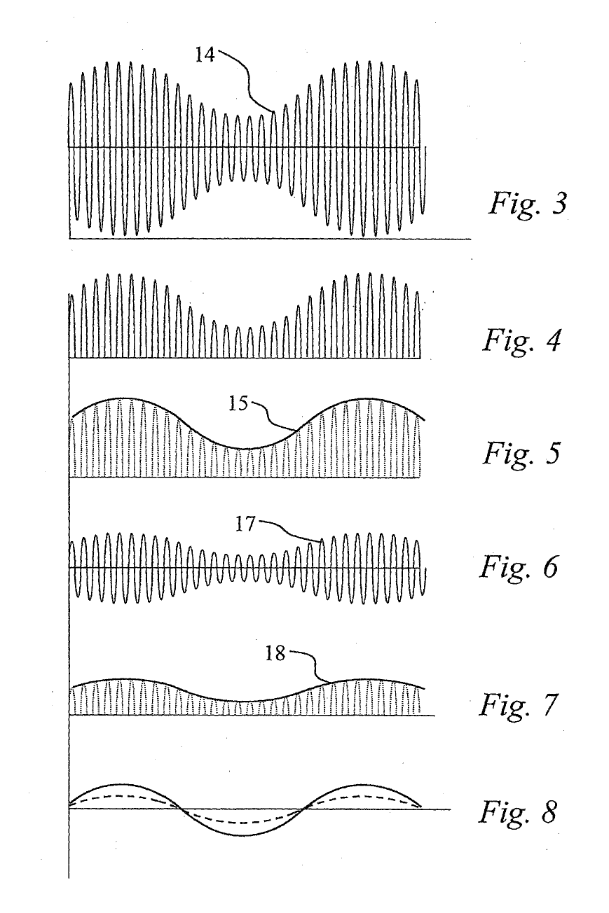

FIGS. 3 to 8 illustrate the signals corresponding to the various phases of the proposed method. FIG. 3 illustrates the wave (carrier) in high frequency 14 (over 10 kHz, preferably 75 kHz or above) measured by electrode 1. After rectifying the half wave, the semi-senoids illustrated in FIG. 4 are obtained, and after filtering, these supply an envelope 15 as per FIG. 5. FIG. 6 illustrates the high-frequency wave 17 measured by electrode 5, which, after rectification and filtering, results in the envelope 18 seen in FIG. 7. FIG. 8 illustrates the overlapping of the envelopes, each line corresponding to the envelope 15 and the dotted line to the envelope 18. An envelope 15 and an envelope 18, which, in this case, correspond to the reference signal, are input to the block 19, whose output is a resulting signal corresponding to the difference between the input signals 15 and 18. It is worth noting that the envelopes 15 and 18 extracted from the signals 14 and 17 are in the illustrated case upper envelopes, and may alternatively be lower envelopes or even a combination of upper and lower envelopes.

Further, it is emphasized that in the proposed method, there is no need for any offsetting of a potential phase difference between the high-frequency signals measured by electrodes 1 and 5, since the signals of interest are the envelopes 15 and 18, which are not affected, or minimally affected, by the phase difference. Furthermore, bearing in mind that the signals of interest are the envelopes and not the carrier, the frequency thereof may be adapted to the patient's conditions or physical constitution so as to optimize the system operation.

The signal injected in the patient can be generated by means of known circuits, such as a single-ended or bipolar current source. Furthermore, a single source may be used for the entire system, or individual sources for each electrode, in which case, the switching elements between the source and each electrode will be dispensed with.

In the diagram in FIG. 2, each signal treatment channel signal 13 or 16 receives the signal from a single electrode, respectively, 1 or 5. However, each channel may receive the signal of more than one electrode, the additional signal being the one coming from a reference electrode, the subtraction between the envelopes being carried out in the channel itself.

Further in connection with the rectification and filtering circuit, an auxiliary circuit may be provided to produce an offset in the diode 13a by applying a continuous positive or negative pre-polarization voltage so as to make the diode operate in the active region (in the case of positive pre-polarization), enabling the rectification of low-amplitude signals.

Signal demodulation can, therefore, be understood as the process of extracting the envelopes (e.g., 15 and 18) from the signals (e.g., 14 and 17) measured in the electrodes (e.g., 1 and 5). It consists, for example, of rectifying the signal measured optionally followed by filtering.

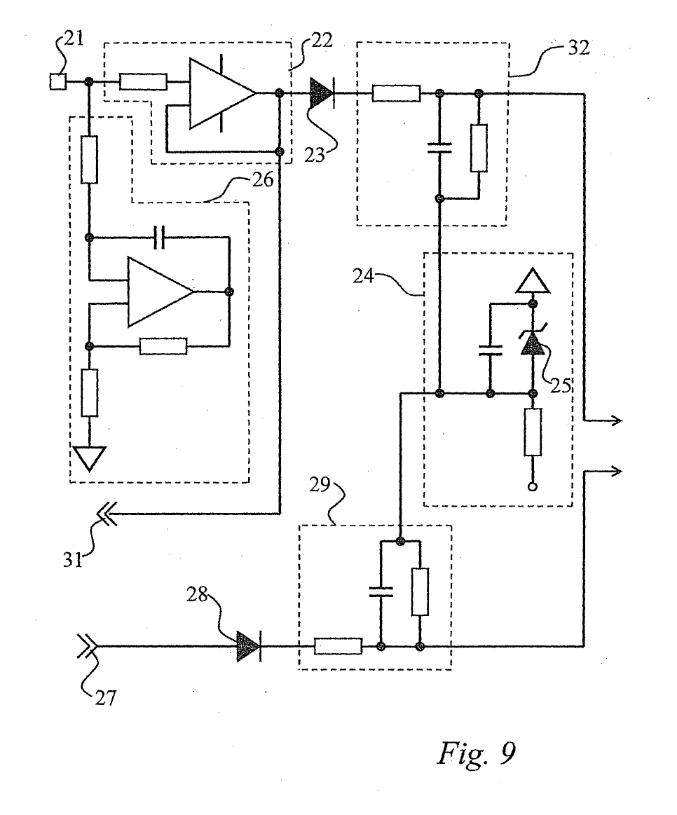

In an alternative or optional form of the disclosure, the fixed capacitances of the input circuit may be offset by using a Negative Impedance Converter—NIC. These may be input capacitances of the operational amplifier or those associated with the analog switch. FIG. 9 exemplifies the use of a circuit endowed with a NIC 26 stage, associated with the input circuit 22, a buffer with high-input impedance for processing the signal measured by the electrode 21 in electrical contact with the patient. The input buffer 22 supplies the signal that will be rectified by the diode 23, as well as a signal present in terminal 31, that may be used as reference signal for another channel. This drawing also shows the presence of a pre-polarization circuit 24 of the diode 23, which introduces an offset determined by the voltage of the reference diode 25. The terminal 27 is the point of introduction of a reference signal already bufferized, which will be rectified by the diode 28 and filtered by the subset 29, so as to supply a low-frequency signal that will be sent to the subtraction circuit (not illustrated) jointly with the low-frequency signal coming from the filter 32, which corresponds to an envelope of the high-frequency signal captured by the electrode 21.

1. A method for acquiring signals for electrical impedance tomography, the method comprising:
injecting an alternating AC signal into at least one electrode positioned on a body of a patient in a region of interest;
measuring a signal induced in the at least one electrode;
extracting at least an envelope from the signal measured; and
determining a difference between the at least an envelope and a reference signal.

2. The method of claim 1, wherein extracting the at least an envelope includes signal demodulation obtained of the measured signal from the at least one electrode.

3. The method of claim 2, wherein the reference signal is an extracted envelope corresponding to a demodulated signal from another electrode.

4. The method of claim 1, wherein the reference signal is the injected alternating AC signal.

5. The method of claim 1, wherein the reference signal one of a fixed voltage value or ground.

6. (canceled)

7. The method of claim 1, wherein the injected AC signal varies in frequency.

8. The method of claim 1, wherein injecting the alternating AC signal into at least one electrode includes injecting the alternating AC signal into more one electrode at the same time.

9. The method of claim 1, wherein injecting the alternating AC signal into at least one electrode includes injecting the alternating AC signal successively into electrodes positioned on the body of the patient to complete a measurement cycle around the region of interest.

10. (canceled)

11. The method of claim 1, wherein determining the difference between the at least envelope and the reference signal is performed by subtracting the reference signal from the envelope through at least one of an electronic analog circuit or digital signal processing.

12. (canceled)

13. The method of claim 1, wherein measuring a signal induced in the at least one electrode includes measuring signals induced in two or more electrodes simultaneously.

14. An apparatus for acquiring electrical impedance tomography signals, the apparatus comprising:
a current source;
a plurality of electrodes, each electrode associated with a channel;
a demodulation circuit per channel, the demodulation circuit configured to generate a demodulated signal responsive to a current injected into the associated electrode;
a signal treatment circuit configured to generate an output signal by performing analog subtraction between the demodulated signal and a reference signal; and
an analog to digital (A/D) converter configured to convert the output signal from an analog signal to a digital signal for processing by a controller.

15. The apparatus of claim 14, wherein the current source is selected from the group consisting of a single-ended type and a bipolar type.

16. (canceled)

17. The apparatus of claim 14, wherein each electrode has its own corresponding current source per channel.

18. The apparatus of claim 14, further comprising at least one Negative Impedance Converter (NIC) circuit, wherein the fixed capacitances of the apparatus are offset by the at least one Negative Impedance Converter (NIC) circuit.

19. The apparatus of claim 18, further comprising an input circuit including an operational amplifier operably coupled to the electrode, wherein the fixed capacitances are input capacitances of the operational amplifier.

20. The apparatus of claim 18, wherein the fixed capacitances are analog switch capacitances.

21. (canceled)

22. The apparatus of claim 14, wherein the demodulation circuit comprises a rectifier diode and an RC filter.

23. The apparatus of claim 22, wherein the demodulation circuit includes an auxiliary circuit configured to produce an offset for the rectifier diode.

24. The apparatus of claim 23, wherein the offset is a positive offset or a negative offset.

25. (canceled)

26. The apparatus of claim 14, wherein the demodulation circuit of each channel receives signals corresponding to more than one electrode.
